(19) United States
(12) Patent Application Publication
(10) Pub. No.: US 2020/0147372 A1
(43) Pub. Date: May 14, 2020

(54) WEARABLE MEDIUM LOW FREQUENCY TREATMENT DEVICE

(71) Applicant: Jae Hwan Kim, Daegu (KR)

(72) Inventor: Jae Hwan Kim, Daegu (KR)

(21) Appl. No.: 16/245,888

(22) Filed: Jan. 11, 2019

(30) Foreign Application Priority Data
Nov. 14, 2018 (KR) 10-2018-0139957

(51) Int. Cl. A61N 1/36; A61N 1/04

(52) U.S. Cl. CPC A61N 1/36003; A61N 1/36014; A61N 1/0484; A61N 1/0452

(57) ABSTRACT

According to an embodiment, a wearable strap device comprises a glove worn on a user's hand and a conductive pad detachably coupled to the glove. A medium/low frequency current generator may be electrically connected with the conductive pad and configured to output a first medium/low frequency current to the conductive pad according to a signal input by the user to contract a wrist muscle for a preset time.

### CROSS-REFERENCE TO RELATED APPLICATION(S)

This application is based on and claims priority under 35 U.S.C. § 119 to Korean Patent Application No. 10-2018-0139957, filed on Nov. 14, 2018 in the Korean Intellectual Property Office, the disclosure of which is incorporated by reference herein in its entirety.

### TECHNICAL FIELD

Embodiments of the disclosure relate to wearable strap devices for generating medium/low frequency currents.

### DISCUSSION OF RELATED ART

People doing bodyweight workouts in gym tend to try to lift heavier weights or do more reps to bulk up within a short time.

Workout gloves may help such gym lovers meet their such desire by retarding the relaxation of the wrist grip and forearm muscles.

However, normal workout gloves cannot respond to the possibility of pain or injury due to excessive muscular contractions.

### SUMMARY

According to an embodiment, a wearable strap device comprises a glove worn on a user's hand and a conductive pad detachably coupled to the glove. A medium/low frequency current generator may be electrically connected with the conductive pad and configured to output a first medium/low frequency current to the conductive pad according to a signal input by the user to contract a wrist muscle for a preset time.

The conductive pad may include at least one or more electrode pairs.

The wearable strap device may further comprise an electromyogram (EMG) sensor configured to detect an EMG of the wrist and measure a muscular strength and a timer configured to set a time and duration of applying the first medium/low frequency current depending on muscle mass of the wrist. The timer may include a display configured to display a standard time for applying the first medium/low frequency current depending on the muscle mass.

The medium/low frequency current generator may include a first medium/low frequency current generator configured to generate the first medium/low frequency current, a second medium/low frequency current generator configured to generate a second medium/low frequency current, and a controller configured to control the first medium/low frequency current generator and the second medium/low frequency current generator. The first medium/low frequency current may have a frequency ranging from 700 Hz to 1000 Hz, and the second medium/low frequency current has a frequency ranging from 4000 Hz to 5000 Hz.

The medium/low frequency current generator may be configured to apply the first medium/low frequency current to the conductive pad when operating in a first operation mode and alternately and repetitively apply the first medium/low frequency current and a second medium/low frequency current to the conductive pad when operating in a second operation mode.

According to embodiments of the disclosure, the wearable strap device for producing medium/low frequency current may temporarily contract the wrist muscles, assisting the user in achieving his desired weight and reps.

### BRIEF DESCRIPTION OF THE DRAWINGS

A more complete appreciation of the present disclosure and many of the attendant aspects thereof will be readily obtained as the same becomes better understood by reference to the following detailed description when considered in connection with the accompanying drawings, wherein:

FIG. 1 is a view illustrating a wearable strap device for producing medium/low frequency currents according to an embodiment;

FIG. 2 is a view illustrating a medium/low frequency generating pad as shown in FIG. 1;

FIG. 3 is a block diagram illustrating a medium/low frequency generating pad as shown in FIG. 2; and

FIG. 4 illustrates medium/low frequency waveforms produced over time or as per the number of movements.

### DETAILED DESCRIPTION OF EXEMPLARY EMBODIMENTS

Hereinafter, exemplary embodiments of the disclosure will be described in detail with reference to the accompanying drawings. The disclosure, however, may be modified in various different ways, and should not be construed as limited to the embodiments set forth herein. When determined to make the subject matter of the present invention unclear, the detailed of the known functions or configurations may be skipped. The same reference denotations may be used to refer to the same or similar elements throughout the specification and the drawings.

As used herein, when an element is “connected” with another element, the element may be “directly connected” with the other element, or the element may be “electrically connected” with the other element via an intervening element.

When an element “includes” another element, the element may further include the other element, rather excluding the other element, unless particularly stated otherwise.

Exemplary embodiments of the disclosure are described below in detail with reference to the accompanying drawings.

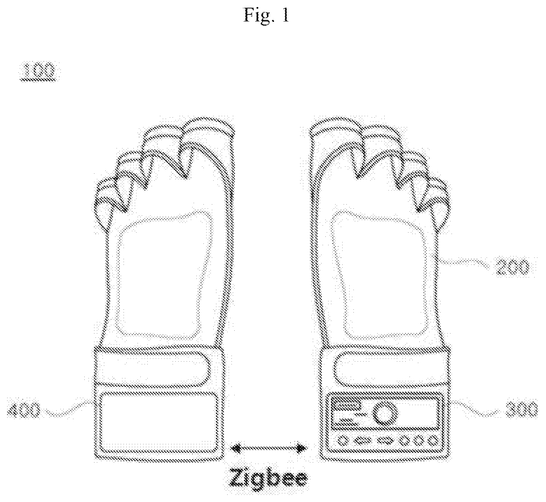

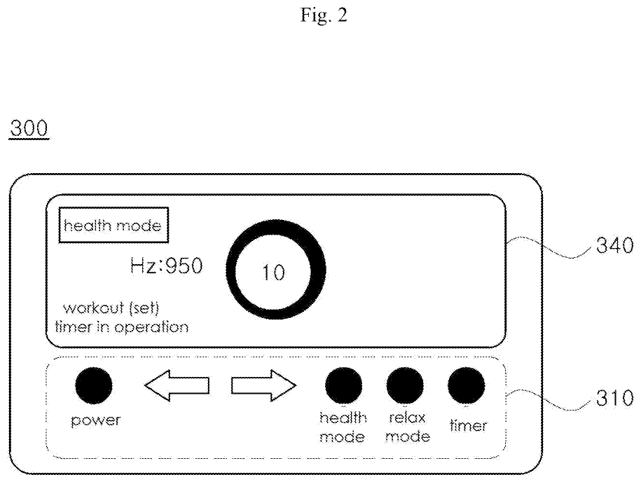

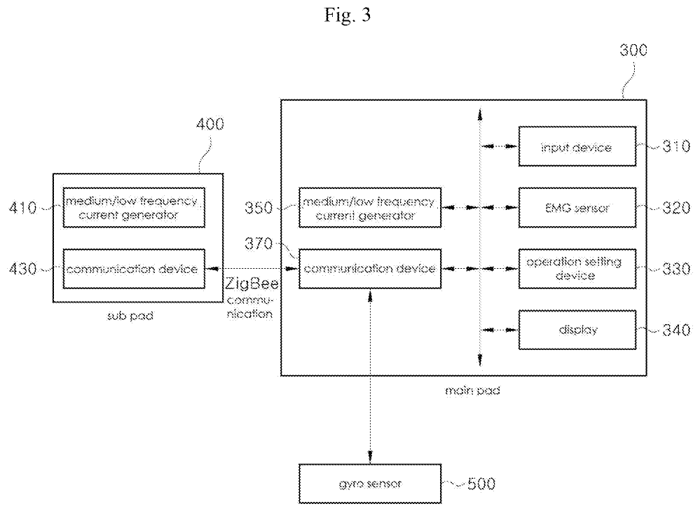

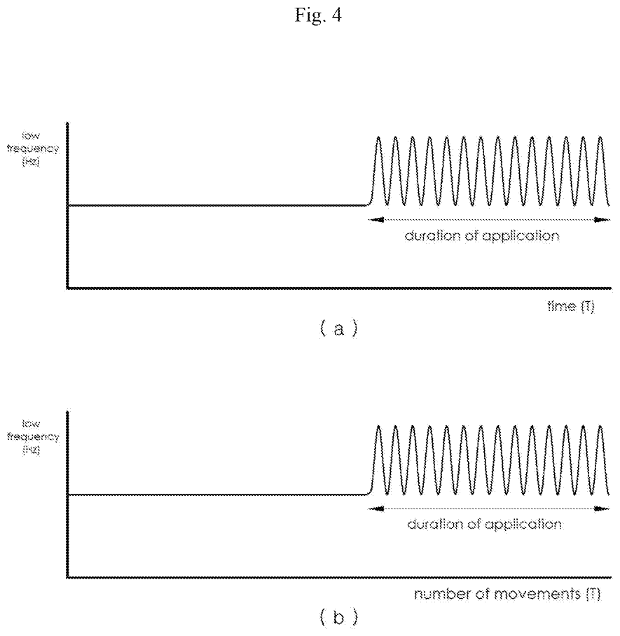

FIG. 1 is a view illustrating a wearable strap device for producing medium/low frequency currents according to an embodiment. FIG. 2 is a view illustrating a medium/low frequency generating pad as shown in FIG. 1. FIG. 3 is a block diagram illustrating a medium/low frequency generating pad as shown in FIG. 2. FIG. 4 illustrates medium/low frequency waveforms produced over time or as per the number of movements.

Referring to FIGS. 1 and 2, a wearable strap device 100 for generating medium/low frequency currents may include a pair of gloves 200 and a pair of medium/low frequency generating pads 300 and 400.

The gloves 200 may be formed to cover or wrap around the user's palms and wrists and may include straps to wrap around the user's palms or wrists.

The gloves 200 may include Velcro tapes to have the medium/low frequency generating pads 300 and 400 attached thereto.

The medium/low frequency generating pads 300 and 400 may be attached to or detached from the gloves 200 via, e.g., Velcro tapes.

The medium/low frequency generating pads 300 and 400 may be, e.g., conductive pads 300 and 400, which are placed on the user's wrists. Although in the embodiments set forth herein the medium/low frequency generating pads 300 and 400 are provided in the form of pads including components shown in FIG. 3 and described below, embodiments of the disclosure are not limited thereto. For example, each medium/low frequency generating pad 300 and 400 may include a separate conductive pad for attachment to the glove 200.

Each conductive pad 300 or 400 may include at least one or more pairs of electrodes (e.g., a positive (+) electrode and a negative (−) electrode) to receive medium/low frequency currents produced from medium/low frequency current generators 410 or 350 and to apply medium/low frequency stimulation. The electrodes may be arranged adjacent each other.

The pair of medium/low frequency generating pads 300 and 400 may be mirrored with each other via Zigbee communication (short-range communication) to simultaneously apply medium/low frequency currents to the conductive pads.

The medium/low frequency generating pads 300 and 400 may include a main pad 300 and a sub pad 400. The main pad 300 and the sub pad 400 are described below in detail for their configuration.

Referring to FIGS. 2 and 3, the main pad 300 may include an input device 310, an electromyogram (EMG) sensor 320, an operation setting device 330, a display 340, a medium/low frequency current generator 350, and a Zigbee communication device 360.

The input device 310 may include at least one or more buttons to power on or off, select a mode (e.g., a health mode or a relax mode), and set a timer (e.g., a current application time and application duration).

The EMG sensor 320 may detect the EMG of the user's wrist through the conductive pad of the glove 200. The EMG sensor 320 may detect the EMG of the wrist and measure the muscular strength of the muscles in the forearm and skeletal muscles. The measured muscular strength may play a role as a factor to determine the magnitude of medium/low frequency current.

The operation setting device 330 may select an operation mode of the medium/low frequency current generator 350 and set any one of the medium/low frequency application time and duration.

The display 340 may display the operation mode of the medium/low frequency current generator, the magnitude of medium/low frequency current, and the medium/low frequency application time and duration.

The medium/low frequency current generator 350 may produce a current, signal, or wave of 4400 Hz or less. The medium/low frequency current generator 350 may produce a low-frequency current and a medium-frequency current depending on operation modes.

The medium-frequency current may be output when the health mode is selected, and the low-frequency current may be output when the relax mode is selected. The low-frequency current may have a frequency not more than 1000 Hz, and the medium-frequency current may have a frequency ranging from 1000 Hz to 4500 Hz.

The low-frequency current may be similar to biometric current that flows in the human body and be used for stimulating muscles and nerves to relieve fatigue, and the medium-frequency current may be used to stimulate muscles to contract.

The medium/low frequency current generator 350 may produce the outputs based on information about the muscular strength of forearm and skeletal muscles measured as per the user's wrist EMG detected by the EMG sensor 320.

The muscular strength information may have, or be set to, one of at least one or more standard muscular strength levels. For example, the muscular strength of the wrist may differ per user, and thus, a different low-frequency current may be required to be applied to each user.

The medium/low frequency current generator 350 may produce and output a low-frequency current and a medium-frequency current matching the muscular strength level of the user's wrist.

The Zigbee communication device 360 may communicate with a Zigbee communication device 430 of the sub pad 400.

The main pad 300 may be connected to an external device (not shown) via a connecting device, such as a communication unit, a connector, or an earphone jack. Such external device may include one of various devices detachably coupled to the main pad 300 to establish a wired connection, e.g., an earphone, an external speaker, a universal serial bus (USB) memory, a charger, a cradle/dock, a digital media broadcast (DMB) antenna, a mobile payment-related device, a health-care device (e.g., a blood glucose meter), or a game player. The external device may include, but is not limited to, a Bluetooth communication device, a near-field communication (NFC) device, or such a short-range communication device, a wireless-fidelity (Wi-Fi) or Wi-Fi Dirct communication device, or a wireless access point (AP) that may be connected to the device 100. The external device may include, but is not limited to, another device, a mobile phone, a smartphone, a tablet PC, a desktop PC, or a server.

The connector may be used as an interface to connect the main pad 300 with an external device (not shown) or a power source (not shown). The main pad 300 may transmit data stored in a storage unit (not shown), or receive data, to/from an external device (not shown) via a cable connected to the connector under the control of the operation setting device 330. The main pad 300 may receive power from a power source (not shown) or charge a battery (not shown) with the power source through the cable connected with the connector.

The sub pad 400 may apply medium/low frequency current to the conductive pad electrically connected thereto based on setting information configured by the main pad 300.

The sub pad 400 may include a medium/low frequency current generator 410 and a Zigbee communication device 420.

The medium/low frequency current generator 410 may provide a current of 4500 Hz or less frequency based on the setting information received from the main pad 300.

The setting information may include the operation modes and medium/low frequency current application time and duration.

According to an embodiment, the wearable strap device 100 may further include a gyro sensor 500.

The gyro sensor 500 may be formed to be detachably attached to a piece of fitness equipment to sense the upper, lower, left, and right movement of the fitness equipment.

The gyro sensor 500 may transmit movement information about the piece of fitness equipment to the main pad 300 through Zigbee communication.

The main pad 300 may set the time of application of low-frequency current based on the movement information.

For example, where the user does bench press with a set of 10 reps, the main pad 300 may make a setting to apply low-frequency current when the user does the tenth rep.

The wearable strap device 100 for generating medium/low frequency currents may delay the time when the user's grip or forearm muscles relax during the course of exercise in gym and thus allows the user to temporarily add weights and reps, giving more satisfaction to the user.

According to an embodiment, a wearable strap device 100 may include a glove 200 worn on a user's hand and a conductive pad detachably coupled to the glove 200. A medium/low frequency current generator 350 or 410 may be electrically connected with the conductive pad and configured to output a first medium/low frequency current to the conductive pad according to a signal through an input device 310 input by the user to contract a wrist muscle for a preset time. The conductive pad may be provided separately from the main pad 300 or the sub pad 400 and be attached to the main pad 300 or the sub pad 400.

According to an embodiment, the conductive pad may include at least one or more electrode pairs as described above.

According to an embodiment, the wearable strap device may include an electromyogram (EMG) sensor 32 configured to detect an EMG of the wrist and measure a muscular strength and a timer configured to set a time and duration of applying the first medium/low frequency current depending on muscle mass of the wrist. The timer may be included in or be provided separately from the input device 310 and may include a display configured to display a standard time for applying the first medium/low frequency current depending on the muscle mass. The display of the timer may be the display 340 or may be a separate display from the display 340.

According to an embodiment, the medium/low frequency current generator 350 or 410 may include a first medium/low frequency current generator configured to generate the first medium/low frequency current, a second medium/low frequency current generator configured to generate a second medium/low frequency current, and a controller configured to control the first medium/low frequency current generator and the second medium/low frequency current generator. The first medium/low frequency current may have a frequency ranging from 700 Hz to 1000 Hz, and the second medium/low frequency current has a frequency ranging from 4000 Hz to 5000 Hz, or more narrowly ranging from 4000 Hz to 4500 Hz.

According to an embodiment, the medium/low frequency current generator 350 or 410 may be configured to apply the first medium/low frequency current to the conductive pad when operating in a first operation mode and alternately and repetitively apply the first medium/low frequency current and a second medium/low frequency current to the conductive pad when operating in a second operation mode. The first operation mode may be the health mode, and the second operation mode may be the relax mode, or the first operation mode may be the relax mode, and the second operation mode may be the health mode.

Although the disclosure has been shown and described in connection with exemplary embodiments thereof, it will be appreciated by one of ordinary skill in the art that various changes or modifications may be made thereto without departing from the scope of the disclosure.

What is claimed is:

1. A wearable strap device, comprising:
a glove worn on a user's hand; and
a conductive pad detachably coupled to the glove, wherein a medium/low frequency current generator is electrically connected with the conductive pad and configured to output a first medium/low frequency current to the conductive pad according to a signal input by the user to contract a wrist muscle for a preset time.

2. The wearable strap device of claim 1, wherein the conductive pad includes at least one or more electrode pairs.

3. The wearable strap device of claim 1, further comprising: an electromyogram (EMG) sensor configured to detect an EMG of the wrist and measure a muscular strength; and a timer configured to set a time and duration of applying the first medium/low frequency current depending on muscle mass of the wrist, wherein the timer includes a display configured to display a standard time for applying the first medium/low frequency current depending on the muscle mass.

4. The wearable strap device of claim 3, wherein the medium/low frequency current generator includes a first medium/low frequency current generator configured to generate the first medium/low frequency current, a second medium/low frequency current generator configured to generate a second medium/low frequency current, and a controller configured to control the first medium/low frequency current generator and the second medium/low frequency current generator, and wherein the first medium/low frequency current has a frequency ranging from 700 Hz to 1000 Hz, and the second medium/low frequency current has a frequency ranging from 4000 Hz to 5000 Hz.

5. The wearable strap device of claim 1, wherein the medium/low frequency current generator is configured to apply the first medium/low frequency current to the conductive pad when operating in a first operation mode and alternately and repetitively apply the first medium/low frequency current and a second medium/low frequency current to the conductive pad when operating in a second operation mode.
